(19) United States
(12) Patent Application Publication
(10) Pub. No.: US 2006/0239939 A1
(43) Pub. Date: Oct. 26, 2006

(54) Deodorant composition

(75) Inventors: Tadahiro Hiramoto, Hiratsuka-shi Kanagawa (JP); Yasutaka Mishima, Hiratsuka-shi (JP); Taichi Yamamoto, Hiratsuka-shi (JP); Tomas Tage Hansen, Bagsvaerd (DK); Kyoko Abe, Chiba-shi (JP)

Correspondence Address: SUGHRUE MION, PLLC, 2100 PENNSYLVANIA AVENUE, N.W., SUITE 800, WASHINGTON, DC, 20037, US

(73) Assignees: Takasago International Corporation, Tokyo (JP); Novozymes Aktieselsxab, Bagsvaerd (DK)

(21) Appl. No.: 10/533,340

(22) PCT Filed: Oct. 31, 2003
(86) PCT No.: PCT/JP03/14006

§ 371 Date: Feb. 6, 2006

(30) Foreign Application Priority Data
Nov. 1, 2002 (JP) 2002-319610

(51) Int. Cl. A61K 8/96

(52) U.S. Cl. 424/50; 424/442; 424/65

(57) ABSTRACT

It is intended to provide deodorant compositions which are nondetrimental to the environment and yet exhibit an excellent deodorizing effect on a wide range of offensive odor components including lower fatty acids. It is also intended to provide deodorant compositions giving off little or no foul odor derived from the substrate as time passes, which is different from the offensive odor to be deodorized. This is accomplished with deodorant compositions containing lignin and an enzyme which oxidizes phenolic compounds. Preferable examples of the lignin include water-soluble lignosulfonic acid and its salt. These compositions may further contain a fragrance and/or a flavor.

### TECHNICAL FIELD

This invention relates to deodorant compositions which contain lignin and a phenolic compound-oxidizing enzyme. More specifically, it relates to deodorant compositions containing a soluble lignin and a phenolic compound-oxidizing enzyme, in particular, deodorant compositions containing water-soluble lignin and a phenolic compound-oxidizing enzyme. Still more specifically, it relates to deodorant compositions to be used for eliminating or relieving odors occurring in our daily life such as halitosis, body odor, odors of bleaching agents or perming liquid, odors in refrigerators and fecal odor and odors in industrial plants and odors of industrial effluent. The invention further relates to oral care products, sanitary products, pet products, foods and animal feeds containing these deodorant compositions.

### BACKGROUND ART

People have been bothered by offensive odors over a long time. As main examples of offensive odor components, there have been known nitrogen-containing compounds such as ammonia, indole, scatole and amines, sulfur-containing compounds such as methylmercaptan, hydrogen sulfide and dimethyl sulfide and lower fatty acids such as butyric acid and isobutyric acid. These components affect humans either separately or in combination. Moreover, there have been pointed out various offensive odors, for example, odors generated from human bodies such as halitosis and body odor, odors of chemicals such as bleaching agents or perming liquids, odors in specific housing areas such as refrigerators, kitchens and closets, fecal odors from diapers, toilets and pet cages and odors in factories and odors of industrial effluent.

With the recent diversification of life styles and changes in consciousness, more and more attention has been paid to various offensive odors in our life and thus we have become sensitive to a wide variety of odors.

Various attempt shave been made to eliminate these offensive odors. For example, it has been known for a long time that polyphenols such as catechins can be used as deodorant components. Also, it is reported that various plant extracts are used as deodorants. By the methods of using polyphenols or deodorants containing the plant extracts, however, offensive odors cannot be eliminated to a satisfactory level.

As deodorants superior in the deodorizing ability to the deodorants as cited above, there have been reported deodorant compositions containing phenolic compounds and phenolic compound-oxidizing enzymes and deodorant compositions containing specific plant extracts with phenolic compound-oxidizing enzymes (see, for example, JP-A-H09-38183, JP-A-H10-212221, JP-A-H03-5484) (the term “JP-A” as used herein means an unexamined published Japanese patent application).

It is true that these deodorant compositions containing enzymes have excellent effects of, for example, eliminating offensive odors over a wide scope, being nondetrimental to the environment and achieving the deodorization by a relatively simple operation. As the results of further discussion on these enzyme-containing deodorant compositions, however, it is clarified that these deodorant compositions are somewhat poor in the effect of eliminating the odors of lower fatty acids such as butyric acid. Although these deodorant compositions have a somewhat poor deodorizing effect on lower fatty acids, it is needless to say that they are still superior in the deodorizing effect on lower fatty acids to other publicly known deodorants.

It was also found that the deodorants containing plant extracts and enzymes give off a slight foul odor derived from the substrate after a long period of time, which is different from the offensive odor.

### DISCLOSURE OF INVENTION

Under these circumstances, an object of the invention is to provide deodorant compositions which are nondetrimental to the environment and yet exhibit an excellent deodorizing effect over a wide range of offensive odor components including lower fatty acids. Another object of the invention is to provide deodorant compositions giving off little or no foul odor from the substrate.

The inventors have conducted intensive studies to solve these problems. As a result, they have found out that a deodorant composition obtained by mixing water-soluble lignin with an enzyme oxidizing a phenolic compound has an effect of eliminating various offensive odors and exerts a highly favorable deodorizing effect on lower fatty acids such as butyric acid and isobutyric acid. Based on this finding, they have continued studies, thereby completing the invention.

Accordingly, the invention relates to:

a deodorant composition characterized by containing lignin and a phenolic compound-oxidizing enzyme;

a deodorant composition as described above wherein the lignin is a soluble lignin;

a deodorant composition as described above wherein the soluble lignin is water-soluble lignin;

a deodorant composition as described above which further contains a fragrance and/or a flavor; and

oral care products, sanitary products, pet products, foods and feeds containing these deodorant compositions.

### BEST MODE FOR CARRYING OUT THE INVENTION

Now, the invention will be described in greater detail.

First, the lignin which is one of the components to be used in the invention will be illustrated. Lignin is a publicly known compound which is contained in a large amount in trees, straw and rice bran and has been frequently studied. Lignin, which is composed of phenylpropane constituting units condensed with each other, typically has a guaiacylpropane structure, a syringylpropane structure, or a p-hydroxyphenylpropane structure. Each of these compounds can be used herein.

In the invention, it is also possible to use a soluble lignin as a substitute for the lignin. The term “soluble lignin” as used herein means lignin which is prepared from lignin and soluble in water, acids or various solvents.

Examples of the soluble lignin include soluble lignin obtained by extracting lignin from a lignin-containing material such as a tree with a solvent by a commonly employed method, the soluble lignin obtained by isolating lignin by a commonly employed method and treating it with an acid or an alkali. Specific examples thereof include alcohol lignin, dioxane lignin, lignin thioglycolate, lignosulfonic acid and its salt, thiolignin, and decomposition products from lignin by oxidation, reduction and hydrolysis.

Among these materials, it is appropriate to use so-called water-soluble lignin which is soluble in ordinary water or water adjusted to be alkaline with a pH value exceeding 7. Typical examples thereof preferably include lignosulfonic acid and its salts which are obtained by reacting isolated lignin with an aqueous sulfite solution at various pH values by the so-called sulfite method, lignin thioglycolate soluble in alkaline water which is obtained by reacting isolated lignin with thioglycolic acid by a commonly employed method, decompositon products from lignin by oxidation which are obtained by reacting lignin with, for example, nitric acid, permanganic acid and its salt, sodium hydroxide and nitrobenzene, decomposition products from lignin by reduction and hydrolysis.

Among all, it is preferable to use lignosulfonic acid or its salts. Namely, it is favorable to use lignosulfonic acid, sodium lignosulfonate or calcium lignosulfonate. Calcium lignosulfonate is particularly favorable because of its high deodorizing activity.

The lignin, soluble lignin and water-soluble lignin as described above can be prepared by publicly known methods.

To avoid complicated discussion, lignosulfonic acid and its salts, which are appropriate examples of the lignin to be used in the invention, will be illustrated in somewhat greater detail.

For example, lignosulfonic acid can be prepared by the sulfite delignification method as follows. Namely, wood chips cut into an appropriate size are immersed in an aqueous solution of sulfurous acid and thermally treated at about 130° C. Thus, lignin in the wood chips is sulfonated and eluted into the liquid reaction mixture. Next, various inorganic substances contained in the liquid reaction mixture are eliminated. Thus, lignosulfonic acid can be obtained.

According to JP-A-H03-5484, a composite offensive odor gas generated at a sewage disposal plant or a human waste-water treatment plant is deodorized by washing with an acid and then treating with a lignosulfonic acid solution. However, this document merely reports treatment with lignosulfonic acid solution and does not report combined use with any enzyme for eliminating the offensive odor. Moreover, effects other than the deodorizing effect achieved by the present invention are not mentioned.

In the present invention, use may be made of a lignosulfonic acid salt or a mixture thereof together with lignosulfonic acid as one of the deodorant components. Also, use may be made of other publicly known phenolic compounds so long as the desired object of the invention can be established thereby.

The soluble lignin specified in the invention falls within the category of so-called monophenolic compounds. Since so-called polyphenolic compounds having a benzene ring substituted at two or more of its hydrogen atoms by hydroxyl groups are employed in many known deodorants, it cannot be anticipated even by those skilled in the art that the soluble lignin (in particular, lignosulfonic acid or its salt) defined in the invention has an excellent deodorizing effect.

The deodorant compositions containing lignosulfonic acid or its salt as one of the components exhibit an excellent deodorizing effect on lower fatty acids such as butyric acid and, furthermore, show an additional effect of giving off little or no foul odor derived from the substrate (in other words, the substance which is changed by the enzymatic reaction). From this viewpoint, it can be also said that the invention cannot be anticipated by those skilled in the art.

Next, the enzyme which is the second component constituting the deodorant composition will be illustrated. Any enzyme can be used herein without particular restriction, so long as it can exhibit the desired deodorizing effect when lignin is also present.

According to the invention three types of oxidoreductases are especially contemplated:

1) Laccases or related enzymes such as tyrosinase cover enzymes which act on molecular oxygen (O₂) and yield water (H₂O) without any need for peroxide (e.g. H₂O₂),

2) Oxidases cover enzymes which act on molecular oxygen (O₂) and yield peroxide (H₂O₂).

3) Peroxidases cover enzymes which act on peroxide (e.g. H₂O₂) and yield water (H₂O).

Preferred oxidoreductases are of microbial, plant or fruit origin. Among these enzymes which are superior in terms of deodorant effect, especially recombinant and/or substantially purified enzymes without any side activity. Microbial enzymes are superior to plant and fruit enzymes as they can be produced more easily in large amounts by recombinant techniques known in the art.

Microbial enzyme means in the context of the present invention enzymes derived from bacteria, filamentous fungi or yeasts.

Furthermore, also genetically modified oxidoreductases designed for the purpose of the invention are contemplated. Said modified oxidoreductases can be selected by screening for oxidoreductases variants having a suitable activity-profile. Said variants may be provided by site directed or cassette or by random mutagenesis by using methods well established in the art.

In the case of enzyme acting on oxygen (O₂) as the acceptor, said oxygen may be molecular oxygen supplied by the air.

Also enzyme systems which comprise a combination of the three types of enzymes are contemplated according to the invention. The enzyme systems may e.g. consist of a laccase or a related enzyme and an oxidase; a laccase or a related enzyme and a peroxidase; a laccase or a related enzyme and an oxidase and a peroxidase; or an oxidase and a peroxidase.

Laccase and related enzymes mentioned in above 1) will be explained.

Examples of specifically contemplated enzymes within the group of laccases and related enzymes which are capable of oxidising VSCs and nitrogen compounds in question are mono- and polyphenol oxidases, such as catechol oxidase (E.C.1.10.3.1), laccase (E.C. 1.10.3.2), tyrosinase (E.C. 1.14.18.1)(E.C. 1.10.3.1), and bilirubin oxidase (E.C. 1.3.3.5). Each of the enzymes is classified under the Enzyme Classification (E.C.) number in accordance with the Recommendations (1992) of the International Union of Biochemistry and Molecular Biology (IUBMB)). E.C. numbers are shown as the combination of alphabets and numbers in parenthesis (and so forth).

Laccase oxidizes o-diphenol as well as p-diphenol forming their corresponding quinones. Tyrosinase or catechol oxidase catalyses two different reactions: The hydroxylation of monophenols in o-diphenols and the oxidation of o-diphenols in o-quinones.

Laccases employed may be derived from a strain of Polyporus sp., in particular a strain of Polyporus pinsitus (also called Trametes villosa) or Polyporus versicolor, or a strain of Myceliophthora sp., e.g. M. thermophila or a strain of Rhizoctonia sp., in particular a strain of Rhizoctonia praticola or Rhizoctonia solani, or a strain of Scytalidium sp., in particular S. thermophilium, or a strain of Pyricularia sp., in particular Pyricularia oryzae, or a strain of Coprinus sp., such as a C. cinereus.

The laccase may also be derived from a fungus such as Collybia, Fomes, Lentinus, Pleurotus, Aspergillus, Neurospora, Podospora, Phlebia, e.g. P. radiata (WO 92/01046), Coriolus sp., e.g. C. hirsitus (JP 2-238885), and Botrytis,

In a preferred embodiment of the invention the laccase is derived from a strain of Myceliophthora sp., especially the Myceliophthora thermophila laccase described in WO 95/33836 (from Novo Nordisk).

Bilirubin oxidase may be derived from a strain of Myrothecium sp., such as a strain of M. verrucaria.

Oxidases and related enzymes mentioned in above 2) will be explained.

Oxidases yielding peroxide (H₂O₂) must be used in combination with a peroxidase to be able to remove or at least reduce malodor.

Suitable oxidases include glucose oxidase (E.C. 1.1.3.4), hexoseoxidase (E.C. 1.1.3.5), L-amino-acidoxidase (E.C. 1.4.3.2), xylitol oxidase, galactose oxidase (E.C. 1.1.3.9), pyranose oxidase (E.C. 1.1.3.10), alcohol oxidase (E.C. 1.1.3.13).

If a L-amino acid oxidase is used it may be derived from a Trichoderma sp. such as Trichoderma harzianum, such as the L-amino acid oxidase described in WO 94/25574 (from Novo Nordisk A/S), or Trichoderma viride.

A suitable glucose oxidase may originate from Aspergillus sp., such as a strain of Aspergillus niger, or from a strain of Cladosporium sp. in particular Cladosporium oxysporum.

Hexose oxidases from the red sea-weed Chondrus crispus (commonly known as Irish moss) (Sullivan and Ikawa, (1973), Biochim. Biophys. Acts, 309, p. 11-22; Ikawa, (1982), Meth. in Enzymol. 89, carbohydrate metabolism part D, 145-149) oxidises a broad spectrum of carbohydrates, such as D-glucose, D-galactose, maltose, cellobiose, lactose, D-glucose 6-phosphate, D-mannose, 2-deoxy-D-glucole, 2-deoxy-D-galactose, D-fucase, D-glucurnic acid, and D-xylose.

Also the red sea-weed Iridophycus flaccidum produces easily extractable hexose oxidases, which oxidise several differentmono- and disaccharides (Bean and Hassid, (1956), J. Biol. Chem, 218, p. 425; Rand et al. (1972, J. of Food Science 37, p. 698-710).

Another suitable group of enzyme is xylitol oxidase which oxidises xylitol, D-sorbitol, D-galactitol, D-mannitol and D-arabinitol in the presence of oxygen. A xylitol oxidase can be obtained from strains of Streptomyces sp. (e.g. Streptomyces IKD472, FERM P-14339). Said enzyme has a pH optimum at 7.5 and is stable at pH 5.5 to 10.5 and at temperatures up to 65° C.

Peroxidases and related enzymes mentioned in above 3) will be explained.

Peroxidases must be used in combination with either H₂O₂ or an oxidase to obtain the desired result, i.e. removal or at least reduction of malodor.

Suitable peroxidases can be found within the group of enzymes acting on peroxide as acceptor, e.g. E.C. 1.11.1, especially peroxidase (E.C. 1.11.1.7).

Specific examples of suitable enzymes acting on peroxide as acceptor include peroxidases derived from a strain of the fungus species Coprinus, in particular a strain of Coprinus cinereus or Coprinus macrorhizus, or derived from a strain of the bacteria genus Bacillus, in particular a strain of Bacillus pumilus.

Haloperoxidases are also suitable according to the invention. Haloperoxidases form a class of enzymes which are able to oxidise halides (Cl-, Br-, I-) in the presence of hydrogen peroxide to the corresponding hypohalous acids. A suitable haloperoxidase is derivable from Curvularia sp., in particular C. verruculosa.

It is possible to use single enzyme described above, however it is also possible to employ the combination of more than 2 enzymes. The enzymes other than those cited above can be used, so long as the desired object can be established thereby.

Each of the enzymes as described above is publicly known per se and can be easily prepared by a known production process. It is also possible to employ a marketed product.

In the deodorant composition of the invention, the content of the enzyme cannot be determined in general since it largely varies depending on, for example, the offensive odor components to be eliminated and the components and form of the deodorant composition. For example, it is preferable to add the enzyme in such an amount as giving an enzyme activity of 100 units or more per 0.1 g of the deodorant composition. The unit of the enzyme activity is defined herein as an increase by 0.001 in the absorptivity at OD 265 nm in case of reacting for 1 minute with the use of (L)-DOPA as a substrate at pH 6.5 and at a temperature of 25° C.

In the invention, the deodorant composition may further contain a fragrance and/or a flavor. As a result, a pleasant smell can be imparted to the deodorant composition. Some of components constituting the deodorant composition would give off a little foul odor characteristic to the substrate. In such a case, the flavor and/or the fragrance can mask the odor to thereby exert a sufficient deodorizing effect.

Although the content of the flavor or the fragrance is not particularly restricted, favorable results can be obtained by using from, for example, 0.01 to 5% by weight thereof based on a soluble lignin.

Preferable examples of the flavor or the fragrance include the following compounds and essential oils: benzyl benzoate, methyl 2-aminobenzoate, methyl 2-((1E)-1-aza-8-hydroxy-4,8-dimethyl-1-nonenyl)benzoate, methyl salicylate, ethyl salicylate, amyl salicylate, hexyl salicylate, benzyl acetate, benzyl salicylate, 1,1,2,3,3-pentamethyl-2,3,5,6,7-pentahydroinden-4-one, 2,6,6,8-tetramethyltricyclo[5.3.1.0(1,5)]undecan-8-ol, citronellol, tricyclo[5.2.1.0(2,6)]-4-decen-8-yl acetate, tricyclo[5.2.1.0(2,6)]-4-decen-8-yl propanoate, 2,6-dimethyloct-7-en-2-ol, 2,6-dimethylheptan-1-ol, phenoxybenzene, 4,6,6,7,8,8-hexamethyl-6,7,8-trihydroxy-cyclopenta[1,2-g]iso chroman, 3-ethoxy-1,1,5-trimethylcyclohexane, hexyl cinnamic aldehyde, isobornyl acetate, 1-(3,4,10,10-tetramethylbicyclo[4.4.0]-5-decen-3-yl)ethan-1-one, 2-((2Z)pent-2-enyl)-3-methylcyclopentane-2-en-1-one, 3-[4-(tert-butyl)phenyl]-2-methylpropanal, linalool, (1E)-1-(6,6-dimethyl-2-methylenecyclohexyl)-1-penten-3-one, 2-noninal-dimethylacetal, 2-phenylethan-1-ol, α-terpineol, 1-((6S,1R)-2,2,6-trimethylcyclohexyl)hexan-3-ol, 2-methoxynaphthalene, 1-(2,6,6,8-tetramethyltricyclo[5.3.1.0(1,5)]-8-undecen-9-yl) ethan-1-one, acetylisoeugenol, allylamyl glycolate, 1,6,10,10-tetramethyl-5-oxatricyclo[7.4.0.0(2,6)]tridecane, (1S,2R,6R)-1,6,10,10-tetramethyl-5-oxatricyclo[7.4.0.0(2,6)]tridecane, amyl-α-cinnamaldehyde, anisaldehyde, 7-methyl-2H,4H-benz[b]1,4-dioxepin-3-one, cinnamyl alcohol, 2-methyl-3-[4-(methylethyl)phenyl]propanal, 2-oxabicyclo[4.4.0]decan-3-one, (2E)-1-(2,6,6-trimethylcyclohexan-3-enyl)butan-2-en-1-one, 2,6-dimethyloctan-7-en-2-ol, 2,6-dimethylheptan-1-ol, eugenol, 2-oxacyclohexadecan-1-one, 3-(4-ethylphenyl)-2,2-dimethylpropanal, geraniol, methyl 2-(3-oxo-2-pentylcyclopentyl)acetate, 2H,4H,4aH,9aH-indano[2,1-d]1,3-dioxane, (3E)-4-(2,6,6-trimethylcyclohexan-2-enylbutan-3-en-2-one, (3E)-4-(6,6-dimethyl-2-methylenecyclohexyl)butan-3-en-2-one, 1-(3,4,10,10-tetramethylbicyclo[4.4.0]-5-decen-3-yl)ethan-1-one, 2-((2Z)-2-pentenyl)-3-methylcyclo-2-penten-1-one, 4-(4-hydroxy-4-methylpentyl)-3-cyclohexene carbaldehyde, 3-[4-(tert-butyl)phenyl]-2-methylpropanal, (5E)-2,6-dimethylheptan-5-enal, methylchavicol, (1E)-1-(6,6-dimethyl-2-methylenecyclohexyl)pentan-1-en-3-one, 2,5-dioxacycloheptadecan-1,6-dione, trans-2-tridecenal, piperanal, thymol, undecalactone, phenylethyl alcohol, phenethyl alcohol, dimethylcyclohexanal, 5-heptyl-3,4,5-trihydrofuran-2-one, 2-(tert-butyl)cyclohexyl acetate, α-fenchyl alcohol, 1-decanal, 2,6-dimethyl-7-octen-2-ol, 4,6,6,7,8,8-hexamethyl-6,7,8-trihydrocyclopenta[1,2-g]isochr oman, 3,7-dimethyl-2,6-octadien-1-al, geranyl nitrile, tetrahydrolinalool, vaniline, caryophyllene, menthol, isopregol, eucalyptol, p-menthane-3,8-diol, vinyl butyl ether, cinnamaldehyde, allyl hexanoate, amyl alcohol, anethol, benzaldehyde, ethylbutyrate, butylbutyrate, isobutylbutyrate, camphor, carvone, pinene, β-caryophyllene, cinnamyl aldehyde, cinnamylalcohol, citral, cumminaldehyde, cymene, decalactone, decanal, diacetyl, ethyl acetoacetate, ethyl anthranate, ethyl hexanoate, ethyl lactate, ethyl 2-methyllactate, ethyl vaniline, ethyl menthol, eugenol, isoeugenol, furfural, furfuryl alcohol, geraniol, hexanal, hexenal, hexyl alcohol, ionone, irone, limonene, linalol, maltol, menthol, menthone, methyl anthranate, methyl cinnamate, nerol, nerolidol, nonalactone, nonanal, octalactone, octanal, octanol, octenol, methyl acetate, propyl acetate, isobutyl acetate, isoamyl acetate, hexyl acetate, hexynyl acetate, octyl acetate, phenyl acetate, benzylacetone, phenylethyl acetate, 1,1-dimethyl-2-phenylethyl acetate, 2-(tert-butyl)cyclohexyl acetate, 4-(tert-butyl)cyclohexyl acetate, linalyl acetate, geranyl acetate, citronellyl acetate, styralyl acetate, anise oil, anise star oil, bergamot oil, basil oil, laurel leaf West Indian oil, galbanum oil, apple oil, apricot oil, cassia oil, camphor tree oil, buchu leaf oil, cardamon seed oil, cassie bark oil, camomile romanoil, cinnamon bark oil, cinnamon leaf oil, clove bud oil, cognac green oil, coriander oil, cubeb oil, caraway oil, fennel oil, garlic oil, ginger oil, petitgrain oil, lemon oil, lime oil, orange oil, citrus oil, cedartree oil, citronella oil, patchouli oil, eucalyptus oil, bay oil, grapefruit oil, mandarin oil, sandalwood oil, juniper berry oil, rose oil, ylang-ylang oil, tangerine oil, geranium oil, limonene, Japanese mint oil and peppermint oil.

Use can be also made of mixtures of two or more of them.

The deodorant composition according to the invention may further contain other components. Examples of these components include fillers such as anhydrous silicates, anhydrous sulfates, various inorganic chlorides, saccharides and polysaccharides, stabilizers, colorants, surfactants, antioxidants such as BHT, BHA, vitamin E and vitamin C, antimicrobial agents such as benzoic acid and sodium benzoate, and marketed deodorants such as active carbon and cyclodextrin. It is also possible to add deodorant compositions containing various plant extracts thereto so long as the desired object can be achieved thereby.

The deodorant composition according to the invention can eliminate or relieve odors offensive to humans. Specific examples of these odors include halitosis, body odor, odor in refrigerators, kitchen odor, garbage odor, odors of various chemicals, odor in factories, odor of industrial effluent, and odor of excreta of animals including humans. In particular, the deodorant composition is excellent in the effect of eliminating body odor based on isobutyric acid, butyric acid and isovaleric acid, kitchen odor involving odors of foods such as fishes and garlic and food putrefaction odor, excreta odors from pet products and diapers, and odors of chemicals such as perming liquids and bleaching agents.

Although it is preferable that the deodorant composition is in the form of a solid powder or granules, the invention is not particularly restricted thereto. Namely, it may be in any form so long as it can exert the deodorizing effect. For example, the deodorant composition may be added to a solvent including water to obtain a solution or a dispersion. Alternatively, it may be held on an arbitrary carrier body to put it into the form of particles or of a sheet. It is also possible to mix the deodorant composition with an arbitrary excipient to obtain a stick or a bar.

Preferable examples of the solvent include water and lower alcohols. Preferable examples of the carrier include saccharides and polysaccharides such as dextrin, cyclodextrin, glucose, lactose and starch, granular materials such as plastic grains and grains of inorganic substances and plastic sheets.

The deodorizing effect of the deodorant composition can be exerted by bringing it into contact with offensive odor components. In case where the deodorant composition is present together with the offensive odors in a solvent, the deodorizing function can be efficiently established.

Although the function mechanism of the deodorizing effect of the deodorant composition according to the invention has not been clarified, it is assumed that the function mechanism of its deodorizing effect on, for example, methylmercaptan might relate to the conversion of phenolic hydroxyl group into quinone and the subsequent formation of thio ether with benzene ring due to the nucleophilic addition of methylthio group.

The deodorant composition according to the invention is efficacious in eliminating or relieving odors over a wide scope. The deodorant composition alone can be employed as a deodorant product. It is also applicable to, for example, various sanitary products, various pet products, bleaching agents, perming liquids and hair care products. Because of being safe to humans and animals, the deodorant composition can be also added to, for example, oral care products such as mouth rinsing solutions, chewing gums and toothpastes, foods, pet foods and livestock feeds.

Now, these products will be illustrated in greater detail.

By adding the deodorant composition of the invention to oral care products, products having a particularly excellent effect of eliminating halitosis can be obtained. For example, it may be added to mouth rinsing solutions, chewing gums or various toothpastes to give products with an excellent effect of eliminating halitosis. The deodorant composition according to the invention is safe to humans, which brings about another merit.

Addition of the deodorant composition of the invention to sanitary products is efficacious in eliminating or relieving offensive odors. That is to say, odors of urine or feces can be eliminated or relieved by preliminarily adding the deodorant composition of the invention to, for example, diapers for infants or aged, bed sheets or mats for bedridden persons, sanitary napkins or pads for incontinence and applying these sanitary products to the parts needed.

Diapers have a fundamental structure composed of a liquid-permeable sheet, a water absorbent and a liquid-inpermeable sheet. The water absorbent is made of water absorbent fibers, water absorbent polymer grains or both in many cases. Urine passing through the liquid-permeable sheet is held by the water absorbent.

It is preferable that the deodorant composition of the invention is preliminarily added to all of the parts constituting diapers as described above. Alternatively, it may be added to a part thereof, for example, the water-absorbent portion alone. In this case where the deodorant composition is added exclusively to the water absorbent portion, it is not always necessary to add the deodorant composition to the whole water absorbent portion. Namely, the deodorant composition may be present exclusively on the surface of the water absorbent or in a part thereof.

The amount of the deodorant composition to be used in the sanitary products cannot be determined in general, since it varies depending on the type of the deodorant composition and the sanitary products. For example, favorable results can be frequently obtained by adding from about 0.01 to 5.0 g of the deodorant composition per gram of the water absorbent substance.

The deodorant composition according to the invention is also usable in pet products. The term pet products as used herein means articles to be applied for various pet animals (i.e., so-called pets) including mammals such as dog, cat, rabbit, Guinea pig and hamster as well as various birds such as parakeet. More specifically speaking, examples of the pet products include pet diapers with increasing consumption in recent years, pet cage sheets and mats, pet toilet sands, pet toilet mats, pet shampoos and pet sanitary products.

By using the deodorant composition of the invention to these pet products, offensive odors can be effectively eliminated or relieved. Namely, the deodorant composition according to the invention may be added and blended with the articles as described above. Alternatively, a layer containing the deodorant composition may be formed on the surface of the articles. When the thus obtained pet products are used in caring for pets, the offensive odors caused by the pets can be efficiently eliminated or relieved.

Because of being safe to humans, the deodorant composition according to the invention can be added to foods. Since the deodorant composition according to the invention per se is odorless, the addition thereof would never damage the inherent odors of foods. However, it should be noted that the deodorant composition according to the invention has a remarkable effect of eliminating offensive odor components such as sulfur-containing compounds and nitrogen-containing compounds. In case of adding the deodorant composition according to the invention to a food containing such compounds which are key components of the smell, it is feared that the balance of the inherent smell of the food per se might be out of balance thereby. Accordingly, it is recommended not to use the deodorant composition according to the invention in such foods.

Moreover, it has been clarified that by taking foods containing the deodorant composition according to the invention, the offensive odor of the feces discharged thereafter can be eliminated or relieved.

The amount of the deodorant composition to be used in the foods cannot be determined in general, since it varies depending on, for example, the components in the foods and the deodorant composition. For example, favorable results can be frequently obtained by adding from about 0.01 to 10% by weight of the deodorant composition per gram of the foods.

Because of being safe to various animals, the deodorant composition according to the invention may be used in feeds including pet foods. The term “feed” as used herein involves cattle, horse and poultry feeds as well as so-called pet foods for dog and cat. Administration of the feeds containing the deodorant composition makes it possible not only to eliminate or relieve the offensive odors of the animals but also to eliminate or relieve the offensive odors of the feces discharged after the administration.

Now, the invention will be described in greater detail by reference to the following Examples. However, it is to be understood that the invention is not construed as being restricted thereto.

### EXAMPLE 1

### Preparation of Deodorant Composition

1 g of calcium lignosulfonate (manufactured by Borregaard Ligno Tech.) was added to 99 g of water and stirred to give a 1% aqueous solution of calcium lignosulfonate. 1 g of tyrosinase (manufactured by SIGMA) was added to 99 g of water and stirred to give a 1% aqueous solution of tyrosinase.

Then the 1% aqueous solution of calcium lignosulfonate was mixed with the same amount of the 1% aqueous solution of tyrosinase. Thus a deodorant composition was obtained.

### EXAMPLE 2

### Preparation of Deodorant Composition

The procedure of Example 1 was followed to give a deodorant composition which was an equal mixture of a 1% aqueous solution of calcium lignosulfonate and a 1% aqueous solution of laccase (manufactured by NOVOZYMES).

### EXAMPLE 3

### Preparation of Deodorant Composition

The procedure of Example 1 was followed to give a deodorant composition which was an equal mixture of a 1% aqueous solution of calcium lignosulfonate and a 1% aqueous solution of laccase (manufactured by DAIWA KAGAKU).

### EXAMPLE 4

### Preparation of Deodorant Composition

The procedure of Example 1 was followed to give a deodorant composition which was an equal mixture of a 1% aqueous solution of calcium lignosulfonate and a 1% aqueous solution of peroxidase (manufactured by SIGMA).

### EXAMPLE 5

### Preparation of Deodorant Composition

10 g of calcium lignosulfonate and 10 g of laccase (manufactured by NOVOZYMES) were mixed together at room temperature to give a powdery deodorant composition which was an equal mixture of calcium lignosulfonate and laccase.

### EXAMPLE 6

### Preparation of Deodorant Composition

The procedure of Example 5 was followed to give a powdery deodorant composition which was an equal mixture of sodium lignosulfonate (manufactured by Borregaard Ligno Tech.) and laccase (manufactured by NOVOZYMES).

### EXAMPLE 7

### Preparation of Deodorant Composition-Containing Stick

The following compounds including the deodorant composition prepared in Example 6 in definite amounts were heated to give a highly viscous solution. Next, this solution was poured into a mold and cooled to give an adiaphoretic stick containing the deodorant composition.

Composition of Adiaphoretic Stick

| | Wt. % |
|---|---|
| PEG-7 glyceryl cocoa acid ester | 2.0 |
| Hydrogenated oil | 5.0 |
| Myristyl myristate | 15.0 |
| Cyclomethicone | 33.0 |
| Stearyl alcohol | 20.0 |
| Stearyl isononenoate | 3.0 |
| Aluminum chlorohydrate | 20.0 |
| Invention product (sodium lignosulfonate:laccase = 1:1 w/w) | 2.0 |
| Total | 100.0 |

### EXAMPLE 8

### Preparation of Deodorant Composition-Containing Mouth Rinsing Solution

The following compounds including the deodorant composition prepared in Example 3 were mixed and thus a mouth rinsing solution was obtained by a method commonly employed.

Composition of Mouth Rinsing Solution:

| | Wt. % |
|---|---|
| Ethyl alcohol | 10.0 |
| Polyoxyethylene hydrogenated castor oil | 2.0 |
| Sodium saccharin | 0.02 |
| Glycerin | 10.0 |
| Sodium benzoate | 0.05 |
| Invention product (calcium lignosulfonate:laccase = 1:1 | 2.0 |
| w/w) | |
| Purified water | the balance |
| Total | 100.0 |

### EXAMPLE 9

### Preparation of Deodorant Composition-Containing Toothpaste

The following compounds including the deodorant composition prepared in Example 6 were mixed and thus a toothpaste was obtained by a method commonly employed.

Composition of Toothpaste:

| | Wt. % |
|---|---|
| Dicalcium phosphate | 10.0 |
| Sodium lauryl sulfate | 2.0 |
| Sodium carboxymethylcellulose | 0.5 |
| Sodium saccharin | 0.02 |
| Sodium benzoate | 10.0 |
| Invention product (sodium lignosulfonate:laccase = 1:1 | 0.1 |
| w/w) | |
| Glycerin | the balance |
| Total | 100.0 |

### EXAMPLE 10

### Preparation of Deodorant Composition-Containing Tablet

The following compounds including the deodorant composition prepared in Example 6 were mixed and thus a tablet of about 6 mm in diameter was obtained by a method commonly employed.

Composition of Tablet:

| | Wt. % |
|---|---|
| Starch | 97.5 |
| Sucrose fatty acid ester | 0.5 |
| Invention product (sodium lignosulfonate:laccase = 1:1 w/w) | 2.0 |
| Total | 100.0 |

### EXAMPLE 11

### Preparation of Deodorant Composition-Containing Chewing Gum

The following compounds including the deodorant composition prepared in Example 6 were mixed and thus a chewing gum was obtained by a method commonly employed.

Composition of Chewing Gum:

| | Wt. % |
|---|---|
| Gum dough | 20.0 |
| Sugar powder | 64.9 |
| Corn starch | 12.5 |
| Acidifying agent | 0.6 |
| Invention product (sodium lignosulfonate:laccase = 1:1 w/w) | 2.0 |
| Total | 100.0 |

### EXAMPLE 12

### Preparation of Deodorant Composition-Containing Dog Food

1.5 g of the deodorant composition prepared in Example 5 was well mixed with 300 g of a dog food (Pedigree Chum Beef: manufactured by Master Foods Ltd.) to give a dog food.

### EXAMPLE 13

### Preparation of Deodorant Composition-Containing Powdery Detergent

The following compounds involving the deodorant composition prepared in Example 6 were mixed and thus a powder detergent was obtained by a method commonly employed.

Composition of Powder Detergent:

| | Wt. % |
|---|---|
| C-12-C-18 sodium valeryl sulfate | 15.0 |
| Sodium carbonate | 15.0 |
| Sodium metasilicate | 13.0 |
| Sodium citrate | 15.0 |
| Carboxymethylcellulose | 2.0 |
| Sodium sulfate | 38.0 |
| Invention product (sodium lignosulfonate:laccase = 1:1 w/w) | 2.0 |
| Total | 100.0 |

### EXAMPLE 14

### Preparation of Deodorant Composition-Containing Shampoo

The following compounds involving the deodorant composition prepared in Example 3 were mixed and thus a shampoo was obtained by a method commonly employed.

Composition of Shampoo:

| | Wt. % |
|---|---|
| Sodium lauryl sulfate | 40.0 |
| Sodium cocoamphoacetate | 10.0 |
| Cocoamido DEA | 2.0 |
| Butylene glycol | 2.0 |
| Citric acid | 0.35 |
| Sodium chloride | 0.1 |
| Methylparaben | 0.2 |
| Propylparaben | 0.1 |
| EDTA tetrasodium | 0.1 |
| Green floral type fragrance | 0.5 |
| Invention product (calcium lignosulfonate:laccase = 1:1 | 2.0 |
| w/w) | |
| Purified water | the balance |
| Total | 100.0 |

### EXAMPLE 15 AND COMPARATIVE EXAMPLES 1 TO 3

### Deodorizing Effect on Lower Fatty Acid Odor

Into a 50 mL vial were successively introduced 1 mL of a 1% aqueous solution of calcium lignos ulfonate (i.e., a component constituting the deodorant composition of Example 1), 40 uL of isobutyric acid and 1 mL of a 1% aqueous solution of tyrosinase (i.e., a component constituting the deodorant composition of Example 1). After covering with a parafilm, the contents of the vial were stirred at 25° C. After stirring for 10 minutes, 50 ml of the head space gas in the vial was passed through a gas detection tube (manufactured by GASTEC) and the concentration of isobutyric acid which was an offensive odor component remaining in the gas was measured. Applying the obtained value to the following formula, the deodorization ratio of the deodorant composition was calculated. In Comparative Examples, the deodorization ratios of a composition free from tyrosinase (manufactured by SIGMA) (Comparative Example 2) and another composition free from calcium lignosulfonate (Comparative Example 3) were also calculated.

Table 1 shows the obtained results.

The control was prepared by adding 2 mL of water as a substitute for 2 mL of the deodorant composition of Example 1.
Deodorization ratio (%)=100×{1−(A)/(B)}

In this formula, A stands for the concentration of the offensive odor components thus measured, while B stands for the concentration of the offensive odor components measured in a control.

TABLE 1

| | | Measured | Deodorization |
|---|---|---|---|
| | | (ppm) | ratio (%) |
| COMP. Ex. 1 | Water alone (control) | 50 | — |
| COMP. Ex. 2 | Calcium lignosulfonate | 35 | 30 |
| | alone | | |
| COMP. Ex. 3 | Tyrosinase alone | 40 | 20 |
| Ex. 15 | Calcium lignosulfonate + | 0 | 100 |
| | tyrosinase | | |

In the above table, the expression “calcium lignosulfonate alone” means a composition to be introduced into a 50 mL vial which was prepared by the same method as in Example 15 but adding 1 ml of water as a substitute of 1 mL of the 1% aqueous solution of tyrosinase.

The expression “tyrosinase alone” means a composition to be introduced into a 50 mL vial which was prepared by the same method as in Example 15 but adding 1 ml of water as a substitute of 1 mL of the 1% aqueous solution of calcium lignosulfonate (the same will apply hereinafter).

### EXAMPLE 16 AND COMPARATIVE EXAMPLES 4 TO 10

### Deodorizing Effect on Lower Fatty Acid Odor

The concentration of an offensive odor component was measured as in Example 15 using however the deodorant composition of Example 2 as a substitute for the deodorant composition of Example 1 employed in Example 15 to thereby evaluate the deodorizing effect of the deodorant composition on the lower fatty acid. Based on the measured value, the deodorization ratio was calculated as in Example 15. In Comparative Examples, the deodorization ratios were also calculated for compositions containing chlorogenic acid alone (Comparative Example 5), a green tea extract alone (Comparative Example 6), a mushroom extract alone (Comparative Example 7), calcium lignosulfonate alone (Comparative Example 8) and laccase alone (Comparative Example 9), instead of calcium lignosulfonate and laccase. Moreover, the deodorization ratio was calculated in the same manner with the use of chlorogenic acid and laccase (manufactured by NOVOZYMES) as Comparative Example 10. Table 2 summarizes the obtained results.

TABLE 2

| | | Measured | Deodorization |
|---|---|---|---|
| | | (ppm) | ratio (%) |
| COMP. Ex. 4 | Water alone (control) | 50 | — |
| COMP. Ex. 5 | Chlorogenic acid alone | 55 | −10 |
| COMP. Ex. 6 | Green tea extract alone | 40 | 20 |
| COMP. EX. 7 | Mushroom extract alone | 55 | −10 |
| COMP. EX. 8 | Calcium lignosulfonate | 35 | 30 |
| | alone | | |
| COMP. EX. 9 | Laccase alone | 40 | 20 |
| Ex. 16 | Calcium lignosulfonate + | 10 | 80 |
| | laccase | | |
| COMP. EX. 10 | Chlorogenic acid + laccase | 35 | 30 |

In the above table, the expression “chlorogenic acid alone” means a composition introduced into a 50 mL vial which was prepared by adding 1 mL of the 1% aqueous solution chlorogenic acid and 1 mL of water as substitutes for 1 mL of the 1% aqueous solution of calcium lignosulfonate and 1 mL of the 1% aqueous solution of laccase.

The expression “green tea extract alone” means a composition introduced into a 50 mL vial which was prepared by adding 2 ml of the green tea extract (containing 80% by weight of catechins) as a substitute for 1 mL of the 1% aqueous solution of calcium lignosulfonate and 1 mL of the 1% aqueous solution of laccase.

The expression “mushroom extract alone” means a composition to be introduced into a 50 mL vial which was prepared by adding 2 ml of the mushroom tea extract as a substitute for 1 mL of the 1% aqueous solution of calcium lignosulfonate and 1 mL of the 1% aqueous solution of laccase.

The expression “laccase alone” means a composition to be introduced into a 50 mL vial which was prepared by adding 1 mL of water as a substitute for 1 mL of the 1% aqueous solution of calcium lignosulfonate.

The expression “chlorogenic acid+laccase” means a composition to be introduced into a 50 mL vial which was prepared by adding 1 mL of a 1% aqueous solution of chlorogenic acid as a substitute for 1 mL of the 1% aqueous solution of calcium lignosulfonate (the same will apply hereinafter).

The green tea extract and the mushroom extract employed above were marketed products.

### EXAMPLE 17 AND COMPARATIVE EXAMPLES 11 TO 13

### Deodorizing Effect on Lower Fatty Acid Odor

The concentration of an offensive odor component was measured as in Example 15 using however the deodorant composition of Example 4 as a substitute for the deodorant composition of Example 1 employed in Example 15 to thereby evaluate the deodorizing effect of the deodorant composition on the lower fatty acid. Then, the deodorization ratio was calculated as in Example 15.

Table 3 summarizes the obtained results.

TABLE 3

| | | Measured | Deodorization |
|---|---|---|---|
| | | (ppm) | ratio (%) |
| COMP. Ex. 11 | Water alone (control) | 50 | — |
| COMP. Ex. 12 | Calcium lignosulfonate | 35 | 30 |
| | alone | | |
| COMP. Ex. 13 | Peroxidase alone | 40 | 20 |
| EX. 17 | Calcium lignosulfonate + | 20 | 60 |
| | peroxidase | | |

In the above table, the expression “peroxidase alone” means a composition introduced into a 50 mL vial which was prepared by adding 1 mL of water as a substitute for 1 mL of the 1% aqueous solution of calcium lignosulfonate (the same will apply hereinafter).

### EXAMPLE 18 AND COMPARATIVE EXAMPLE S 14 TO 16

### Deodorizing Effect on Lower Fatty Acid Odor

2 g of shavings of the adiaphoretic stick obtained in Example 7 were added to 5 mL of a 0.25% aqueous solution of butyric acid and mixed at room temperature. After 10 minutes, the mixture was sensorily evaluated by 3 skilled panelists in accordance with the following criteria.

For comparison, adiaphoretic sticks containing the components listed in Table 4 were sensorily evaluated in the same manner. (Comparative Examples 14 to 16)

Table 4 summarizes the obtained results.

Criteria for Evaluation:

Score 1) No butyric acid odor.

Score 2) Slight butyric acid odor.

Score 3) Somewhat butyric acid odor.

Score 4) Obvious butyric acid odor.

Score 5) Strong butyric acid odor.

Score 6) Extremely strong butyric acid odor.

TABLE 4

| | | Average score |
|---|---|---|
| COMP. EX. 14 | Control | 6.0 |
| COMP. EX. 15 | Adiaphoretic containing sodium | 3.7 |
| | lignosulfonate | |
| COMP. EX. 16 | Adiaphoretic containing laccase | 6.0 |
| EX. 18 | Adiaphoretic containing sodium | 1.0 |
| | lignosulfonate + laccase | |

In the above table, the control means an adiaphoretic of Example 7 but being free from sodium lignosulfonate+laccase, the adiaphoretic containing sodium lignosulfonate means the adiaphoretic of Example 7 but prepared not using laccase, and the adiaphoretic containing laccase means the adiaphoretic of Example 7 but prepared not using sodium lignosulfonate.

### EXAMPLE 19 AND COMPARATIVE EXAMPLES 17 TO 24

### Deodorizing Effect on Methylmercaptan

Into a 50 mL vial were successively introduced 1 mL of a 1% aqueous solution of calcium lignosulfonate (i.e., a component constituting the deodorant composition of Example 1), 2 uL of a 15% aqueous solution of sodium methylmercaptan and 1 mL of a 1% aqueous solution of tyrosinase (i.e., a component constituting the deodorant composition of Example 1). After covering with a parafilm, the contents of the vial were stirred at 25° C. After stirring for 10 minutes, 50 ml of the head space gas in the vial was passed through a gas detection tube (manufactured by GASTEC) and the concentration of a sulfur-containing compound which is an offensive odor component remaining in the gas was measured. Then, the deodorization ratio was calculated as in Example 15. In Comparative Examples, deodorization ratios were calculated in the same manner using however chlorogenic acid alone (Comparative Example 18), ferulic acid alone (Comparative Example 20) and catechol alone (Comparative Example 21) each as a substitute for a mixture of calcium lignosulfonate and tyrosinase. In other Comparative Examples, the deodorization ratios were calculated in the same manner for a mixture of ferulic acid and tyrosinase (Comparative Example 23) and a mixture of chlorogenic acid and tyrosinase (Comparative Example 24).

Table 5 summarizes the obtained results.

TABLE 5

| | | Measured | Deodorization |
|---|---|---|---|
| | | (ppm) | ratio (%) |
| COMP. Ex. 17 | Water alone (control) | 200 | — |
| COMP. Ex. 18 | Chlorogenic acid alone | 320 | −60 |
| COMP. Ex. 19 | Calcium lignosulfonate | 180 | 10 |
| | alone | | |
| COMP. Ex. 20 | Ferulic acid alone | 280 | −40 |
| COMP. Ex. 21 | Catechol alone | 200 | 0 |
| COMP. Ex. 22 | Tyrosinase alone | 200 | 0 |
| COMP. Ex. 23 | Ferulic acid + tyrosinase | 300 | −50 |
| COMP. Ex. 24 | Chlorogenic acid + | 80 | 60 |
| | tyrosinase | | |
| Ex. 19 | Calcium lignosulfonate + | 0 | 100 |
| | tyrosinase | | |

In the above table, the expression “ferulic acid alone” means an example wherein 1 mL of water was used as a substitute for 1 mL of the 1% aqueous solution of tyrosinase in the composition of Comparative Example 23, the expression “tyrosinase alone” means an example wherein 1 mL of water was used as a substitute for 1 mL of the 1% aqueous solution of calcium lignosulfonate in the composition of Example 19, and the expression “ferulic acid+tyrosinase” means an example wherein ferulic acid and tyrosinase were employed (the same will apply hereinafter).

### EXAMPLE 20 AND COMPARATIVE EXAMPLES 25 TO 27

### Deodorizing Effect on Methylmercaptan

The deodorizing effects on methylmercaptan of compositions were measured as in Example 19 using however the deodorant composition of Example 2 as a substitute for the deodorant composition of Example 1 employed in Example 19.

Table 6 summarizes the obtained results.

TABLE 6

| | | Measured | Deodorization |
|---|---|---|---|
| | | (ppm) | ratio (%) |
| COMP. Ex. 25 | Water alone (control) | 200 | — |
| COMP. Ex. 26 | Calcium lignosulfonate | 160 | 20 |
| | alone | | |
| COMP. Ex. 27 | Laccase alone | 200 | 0 |
| Ex. 20 | Calcium lignosulfonate + | 20 | 90 |
| | laccase | | |

### EXAMPLE 21 AND COMPARATIVE EXAMPLES 28 TO 30

### Deodorizing Effect on Methylmercaptan

The deodorizing effects on methylmercaptan of compositions were measured as in Example 19 using however the deodorant composition of Example 4 as a substitute for the deodorant composition of Example 1 employed in Example 19.

Table 7 summarizes the obtained results.

TABLE 7

| | | Measured | Deodorization |
|---|---|---|---|
| | | (ppm) | ratio (%) |
| COMP. Ex. 28 | Water alone (control) | 200 | — |
| COMP. Ex. 29 | Calcium lignosulfonate | 160 | 20 |
| | alone | | |
| COMP. Ex. 30 | Peroxidase alone | 200 | 0 |
| Ex. 21 | Calcium lignosulfonate + | 120 | 40 |
| | peroxidase | | |

### EXAMPLE 22 AND COMPARATIVE EXAMPLES 31 TO 33

### Deodorizing Effect on Halitosis

Starting with 4 g of garlic and 1 L of water, an extract was prepared. 10 mL of the garlic extract thus obtained was poured into a 50 mL bottle. Then 1 mL of the mouth rinsing solution of Example 8 was added thereto and mixed. Subsequently, the mixture was shaken at 34° C. for 3 minutes. The obtained mixture was sensorily evaluated by 5 skilled panelists in accordance with the following criteria.

Table 8 summarizes the obtained results.

Criteria for Evaluation:

Score 1) No garlic odor.

Score 2) Slight garlic odor.

Score 3) Somewhat garlic odor.

Score 4) Obvious garlic odor.

Score 5) Strong garlic odor.

Score 6) Extremely strong garlic odor.

TABLE 8

| | | Average score |
|---|---|---|
| COMP. EX. 31 | Control | 6.0 |
| COMP. EX. 32 | Mouth rinsing solution containing | 4.2 |
| | calcium lignosulfonate | |
| COMP. EX. 33 | Mouth rinsing solution containing | 6.0 |
| | laccase | |
| EX. 22 | Mouth rinsing solution containing | 1.0 |
| | calcium lignosulfonate + laccase | |

In the above table, the control means a mouth rinsing solution free from calcium lignosulfonate+laccase.

### EXAMPLE 23 AND COMPARATIVE EXAMPLES 34 TO 37

### Deodorizing Effect on Halitosis

The following method was employed to evaluate halitosis deodorizing effects achieved by using deodorant compositions in toothpastes.

After thoroughly rinsing the mouth, each subject held 10 mL of a 50 ppm solution of sodium methylmercaptan in the mouth. After 1 minute, the solution was spitted out. Immediately thereafter, the gas was exhaled by the subject and collected into a 5 L plastic bag.

Next, the subject brushed the teeth for 2 minutes using the tooth paste prepared in Example 9. Immediately thereafter, the gas was exhaled and collected into a 5 L plastic bag.

Then the gas in the plastic bag exhaled after brushing the teeth was evaluated by 4 panelists in accordance with the following criteria while comparing with the gas collected in the plastic bag before brushing the teeth.

Table 9 summarizes the obtained results.

Criteria for Evaluation:

Score 1) No methylmercaptan odor.

Score 2) Slight methylmercaptan odor.

Score 3) Somewhat methylmercaptan odor.

Score 4) Obvious methylmercaptan odor.

Score 5) Strong methylmercaptan odor.

Score 6) Extremely strong methylmercaptan odor.

TABLE 9

| | | Average |
|---|---|---|
| | | score |
| COMP. EX. 34 | Control (1) | 6.0 |
| COMP. EX. 35 | Control (2) | 4.5 |
| COMP. EX. 36 | Toothpaste containing sodium | 3.8 |
| | lignosulfonate | |
| COMP. Ex. 37 | Toothpaste containing laccase | 4.5 |
| EX. 23 | Toothpaste containing sodium | 1.0 |
| | lignosulfonate + laccase | |

In the above table, the control (1) means a case of the gas exhaled by a subject who did not brush his/her teeth while the control (2) means use of toothpaste free from sodium lignosulfonate+laccase.

### EXAMPLE 24 AND COMPARATIVE EXAMPLES 38 TO 41

### Deodorizing Effect on Halitosis

The following method was employed to evaluate the effect of eliminating halitosis by adding a deodorant composition to tablets of about 6 mm in diameter.

After thoroughly rinsing the mouth, each subject held 10 mL of a 50 ppm solution of sodium methylmercaptan in the mouth. After 1 minute, the solution was spitted out. Immediately thereafter, the gas was exhaled by the subject and collected into a 5 L plastic bag.

Next, the subject ingested the tablets prepared in Example 10 for 10 minutes. Immediately thereafter, the gas was exhaled and collected into a 5 L plastic bag.

Then, the gas in the plastic bag exhaled after taking the tablet was evaluated by 4 panelists in accordance with the same criteria as in Example 23 while comparing with the gas collected in the plastic bag before taking the tablet.

Table 10 summarizes the obtained results.

TABLE 10

| | | Average score |
|---|---|---|
| COMP. EX. 38 | Control (1) | 6.0 |
| COMP. EX. 39 | Control (2) | 4.8 |
| COMP. EX. 40 | Tablet containing sodium | 3.5 |
| | lignosulfonate | |
| COMP. Ex. 41 | Tablet containing laccase | 4.8 |
| EX. 24 | Tablet containing sodium | 1.3 |
| | lignosulfonate + laccase | |

In the above table, the control (1) means a case of the gas exhaled by a subject who did not ingest the tablet while the control (2) means a case of tablets free from both sodium lignosulfonate and laccase.

### EXAMPLE 25 AND COMPARATIVE EXAMPLES 42 TO 45

### Deodorizing Effect on Halitosis

The following method was employed to evaluate the effect of eliminating halitosis by using the deodorant composition-containing chewing gum obtained in Example 11.

After thoroughly rinsing the mouth, each subject held 10 mL of a 50 ppm solution of sodium methylmercaptan in the mouth. After 1 minute, the solution was spitted out. Immediately thereafter, the gas was exhaled by the subject and collected into a 5 L plastic bag.

Next, the subject chewed the chewing gum continuously for 10 minutes. After 10 minutes, the gas exhaled was immediately collected into a 5 L plastic bag.

Then the gas in the plastic bag exhaled after continuously chewing the gum was evaluated by 4 panelists in accordance with the same criteria as in Example 23 while comparing with the gas collected in the plastic bag before chewing the gum.

Table 11 summarizes the obtained results.

TABLE 11

| | | Average score |
|---|---|---|
| COMP. EX. 42 | Control (1) | 6.0 |
| COMP. EX. 43 | Control (2) | 4.3 |
| COMP. EX. 44 | Chewing gum containing sodium | 3.3 |
| | lignosulfonate | |
| COMP. Ex. 45 | Chewing gum containing laccase | 4.5 |
| EX. 25 | Chewing gum containing sodium | 1.3 |
| | lignosulfonate + laccase | |

In the above table, the control (1) means a case of the gas exhaled by a subject who did not chew the gum while the control (2) means use of chewing gum free from sodium lignosulfonate+laccase.

### EXAMPLES 26 AND 27 AND COMPARATIVE EXAMPLES 46 to 51

### Application to Paper Pad for Humans

From a marketed paper diaper pad for adults (Nyotori Pad Super L Size™ manufactured by UNICHARM), a water-absorbent polymer (about 1 g) was taken out and 0.5 g of the deodorant composition prepared in Example 5 was added thereto. Then the polymer was returned to the paper pad again. 200 mL of urine of an adult male subject was absorbed by this paper pad which was then sealed in a 500 ml Teddler Bags™ (manufactured by Fine) and maintained at 34° C. for a definite time. Subsequently, the bag was taken out and the odor of the paper pad was sensorily evaluated by 7 panelists in accordance with the following evaluation criteria. Tables 12 and 13 summarize the obtained results.

For comparison, use was also made of the same paper pads but free from the deodorant composition (Comparative Examples 46 and 49), marketed paper pads further containing a deodorant (Powerful Shoshu Acty Nyotori Pad manufactured by Crecia corporation) (Comparative Examples 47 and 50) and paper pads containing 0.5 g of a mixture of a fresh coffee bean extract with laccase (manufactured by DAIWA KAGAKU) at a ratio of 1:1 (w/w)

Criteria for Evaluation (1):

The urine odor level was sensorily evaluated.

Score 1) No urine odor.

Score 2) Slight urine odor.

Score 3) Somewhat urine odor.

Score 4) Obvious urine odor.

Score 5) Strong urine odor.

Score 6) Extremely strong urine odor.

TABLE 12

| | 1 h | 3 h | 6 h | 24 h | 48 h | 72 h |
|---|---|---|---|---|---|---|
| EX. 26 | 1.4 | 1.6 | 2.1 | 2.6 | 2.7 | 3.3 |
| COMP. EX. 46 | 6.0 | 6.0 | 6.0 | 6.0 | 6.0 | 6.0 |
| COMP. EX. 47 | 5.3 | 5.7 | 5.7 | 6.0 | 6.0 | 6.0 |
| COMP. EX. 48 | 2.1 | 2.6 | 2.7 | 3.4 | 3.7 | 3.9 |

Criteria for Evaluation (2):

The total odor level including the urine odor, the foul odor from the substrate and the odor caused by the interaction between them was sensorily evaluated.

Score 1) No overall odor.

Score 2) Slight overall odor.

Score 3) Somewhat overall odor.

Score 4) Obvious overall odor.

Score 5) Strong overall odor.

Score 6) Overall odor unbearable.

TABLE 13

| | 1 h | 3 h | 6 h | 24 h | 48 h | 72 h |
|---|---|---|---|---|---|---|
| EX. 27 | 1.2 | 1.2 | 1.5 | 1.8 | 2.2 | 2.2 |
| COMP. EX. 49 | 5.0 | 5.0 | 5.0 | 5.0 | 5.0 | 5.0 |
| COMP. EX. 50 | 4.3 | 4.8 | 4.7 | 4.9 | 5.0 | 5.0 |
| COMP. EX. 51 | 2.2 | 2.6 | 2.8 | 3.4 | 3.8 | 3.9 |

### EXAMPLES 28 TO 29 AND COMPARATIVE EXAMPLES 52 TO 55

### Deodorizing Effect on Human Urine and Feces

10 ml of adult male urine and 20 mg of the deodorant composition of Example 5 were introduced into a 100 mL vial. After covering with a parafilm (manufactured by American National Can), the mixture was stirred at 25° C. for 10 minutes. Then the contents of the vial were sensorily evaluated by 7 panelists in accordance with the evaluation criteria (1) of Examples 26 and 27.

Table 14 summarizes the obtained results.

For comparison, a case of the urine alone (control, Comparative Example 52), a case where sodium lignosulfonate alone was added to the urine (Comparative Example 53), a case where tyrosinase alone was added to the urine (Comparative Example 54) and a case where a lime fragrance (manufactured by TAKASAGO) alone was added to the urine (Comparative Example 55) are also presented.

TABLE 14

| | | Average score |
|---|---|---|
| COMP. EX. 52 | Urine alone | 6.0 |
| COMP. EX. 53 | Urine + calcium lignosulfonate | 4.3 |
| COMP. EX. 54 | Urine + laccase | 6.0 |
| COMP. EX. 55 | Urine + lime fragrance | 4.7 |
| Ex. 28 | Urine + invention product | 1.3 |
| Ex. 29 | Urine + invention product + lime | 1.0 |
| | fragrance | |

In the above table, the expression “urine+calcium lignosulfonate” means a case where 20 mg of calcium lignosulfonate was added to the urine, the expression “urine+laccase” means a case where 20 mg of laccase was added to the urine, and the expression “urine+lime fragrance” means a case where 10 μl of the lime fragrance was added to the urine. In the case of “urine+invention product+lime fragrance”, 10 μl of the lime fragrance was added.

### EXAMPLE 30 AND COMPARATIVE EXAMPLES 56 TO 57

### Deodorizing Effect on Urine and Feces

Evaluation of the Effect of Eliminating Fecal Odor by Ingesting Deodorant Composition-Containing Feed

Dogs for experimental use were fed with a dog food (Pedigree Chum Beef: manufactured by Master Foods Ltd.) twice a day for 3 days. On the next day, the deodorant composition-containing dog food of Example 12 was given to the animals twice a day. On the next morning, the odor of the discharged feces was sensorily evaluated by 4 panelists by the following evaluation method in accordance with the following evaluation criteria.

Table 15 summarizes the obtained results.

Evaluation Method:

Using the odor of the feces discharged in case of taking the ordinary food as a standard, the odor of the feces on the next day after the administration of the deodorant composition according to the invention was evaluated.

Criteria for Evaluation:

Score 1: Extremely weakened.

Score 2: Weakened.

Score 3: Somewhat weakened.

Score 4: Not changed.

Results:

TABLE 15

| | | Average score |
|---|---|---|
| COMP. EX. 56 | Feed containing calcium lignosulfonate | 3.3 |
| COMP. EX. 57 | Feed containing laccase | 3.5 |
| EX. 30 | Feed containing calcium | 1.0 |
| | lignosulfonate + laccase | |

### EXAMPLE 31 AND COMPARATIVE EXAMPLE 58 TO 60

### Deodorizing Effect on Menstrual Odor

Into a 100 mL vial were introduced 10 mL of vaginal malodor and 20 mL of the deodorant composition of Example 4. After covering with a parafilm, the mixture was incubated by shaking at 25° C. for 10 minutes. Then sensory evaluation was carried out by 7 panelists in accordance with the following evaluation criteria.

Table 16 shows the obtained results.

For comparison, a case using the vaginal malodor alone, a case where 20 mg of calcium lignosulfonate alone was added to the malodor and a case where 20 mg of peroxidase alone was added are also presented.

Criteria for Sensory Evaluation:

Score 1) No odor.

Score 2) Slight odor.

Score 3) Somewhat odor.

Score 4) Obvious odor.

Score 5) Strong odor.

Score 6) Extremely strong odor.

TABLE 16

| | | Average score |
|---|---|---|
| COMP. EX. 58 | vaginal malodor alone | 6.0 |
| COMP. EX. 59 | vaginal malodor + calcium | 4.7 |
| | lignosulfonate | |
| COMP. EX. 60 | vaginal malodor + peroxidase | 6.0 |
| EX. 31 | Invention product | 1.4 |

### EXAMPLE 32 AND COMPARATIVE EXAMPLES 61 TO 63

### Deodorizing Effect on Livestock Feces and Urine

Into a 100 mL vial were introduced 10 mL of a liquid separated from livestock feces and urine and 20 mg of the deodorant composition of Example 3. After covering with a parafilm, the mixture was incubated by shaking at 25° C. for 10 minutes. Then sensory evaluation was carried out by 7 panelists in accordance with the following evaluation criteria.

Table 17 shows the obtained results.

For control, a case using the separated liquid alone, a case where 20 mg of calcium lignosulfonate alone was added to the separated liquid and a case where 20 mg of laccase alone was added to the separated liquid are also presented.

Criteria for Sensory Evaluation:

Score 1) No fecal odor.

Score 2) Slight fecal odor.

Score 3) Somewhat fecal odor.

Score 4) Obvious fecal odor.

Score 5) Strong fecal odor.

Score 6) Extremely strong fecal odor.

TABLE 17

| | | Average score |
|---|---|---|
| COMP. EX. 61 | Separated liquid alone | 6.0 |
| COMP. EX. 62 | Separated liquid + calcium | 4.3 |
| | lignosulfonate | |
| COMP. EX. 63 | Separated liquid + laccase | 6.0 |
| EX. 32 | Invention product | 1.3 |

### EXAMPLE 33 AND COMPARATIVE EXAMPLES 64 TO 66

### Deodorizing Effect on Ammonia

Into a 50 mL vial were successively introduced 1 mL of a 1% aqueous solution of calcium lignosulfonate, 1 mL of a 1% aqueous solution of tyrosinase (constituting the deodorant composition of Example 1) and 5 uL of a 2.8% aqueous ammonia. After covering with a parafilm, the contents of the vial were stirred at 25° C. for ten minutes. Next, 50 mL of the head space gas in the vial was passed through a gas detection tube (manufactured by GASTEC) and the concentration of the offensive odor component remaining in the gas was measured. Then the deodorization ratio was calculated as in Example 15.

Table 18 summarizes the obtained results.

As a control, a case where 2 mL of water alone was substituted for 1 mL of the 1% aqueous solution of calcium lignosulfonate and 1 mL of the 1% aqueous solution of tyrosinase is presented.

For comparison, moreover, a case of calcium lignosulfonate alone where 1 mL of water was added as a substitute for 1 mL of the 1% aqueous solution of tyrosinase and a case of tyrosinase alone where 1 mL of water was added as a substitute for 1 mL of the 1% aqueous solution of calcium lignosulfonate are presented.

TABLE 18

| | | Measured | Deodorization |
|---|---|---|---|
| | | (ppm) | ratio (%) |
| COMP. Ex. 64 | Water alone (control) | 20 | — |
| COMP. Ex. 65 | Calcium lignosulfonate | 10 | 50 |
| | alone | | |
| COMP. Ex. 66 | Tyrosinase alone | 20 | 0 |
| Ex. 33 | Calcium lignosulfonate + | 0 | 100 |
| | tyrosinase | | |

In the above table, the expression “calcium lignosulfonate alone” means an example wherein 1 mL of water was used as a substitute for 1 mL of the 1% aqueous solution of tyrosinase in the composition, while the expression “tyrosinase alone” means an example wherein 1 mL of water was used as a substitute for 1 mL of the 1% aqueous solution of calcium lignosulfonate in the composition (the same will apply hereinafter).

### EXAMPLE 34 AND COMPARATIVE EXAMPLES 67 TO 69

### Deodorizing Effect on Ammonia

The deodorizing effects on ammonia of compositions were measured as in Example 33′ using however the deodorant composition of Example 2 as a substitute for the deodorant composition of Example 1 employed in Example 33.

Table 19 summarizes the obtained results.

TABLE 19

| | | Measured | Deodorization |
|---|---|---|---|
| | | (ppm) | ratio (%) |
| COMP. Ex. 67 | Water alone (control) | 20 | — |
| COMP. Ex. 68 | Calcium lignosulfonate | 10 | 50 |
| | alone | | |
| COMP. Ex. 69 | Laccase alone | 20 | 0 |
| Ex. 34 | Calcium lignosulfonate + | 5 | 75 |
| | laccase | | |

### EXAMPLE 35 AND COMPARATIVE EXAMPLES 70 TO 72

### Deodorizing Effect on Ammonia

The deodorizing effects on ammonia of compositions were measured as in Example 33 using however the deodorant composition of Example 4 as a substitute for the deodorant composition of Example 1 employed in Example 33.

Table 20 summarizes the obtained results.

TABLE 20

| | | Measured | Deodorization |
|---|---|---|---|
| | | (ppm) | ratio (%) |
| COMP. Ex. 70 | Water alone (control) | 20 | — |
| COMP. Ex. 71 | Calcium lignosulfonate | 10 | 50 |
| | alone | | |
| COMP. Ex. 72 | Peroxidase alone | 20 | 0 |
| Ex. 35 | Calcium lignosulfonate + | 5 | 75 |
| | peroxidase | | |

### EXAMPLE 36 AND COMPARATIVE EXAMPLES 73 TO 75

### Deodorizing Effect on Fish Odor

1 g of the deodorant composition prepared in Example 5 was added to 5 L of water and the mixture was stirred well. Then, a pan in which sardines had been cooked was immersed in the mixture. After 10 minutes at room temperature, the pan was taken out and the deodorant composition-containing solution was washed away with water. Subsequently, the occurrence of the odor and the level thereof on the pan surface were sensorily evaluated by 5 skilled panelists in accordance with the following evaluation criteria.

Table 21 summarizes the obtained results.

Criteria for Evaluation:

Score 1) No fish odor.

Score 2) Slight fish odor.

Score 3) Somewhat fish odor.

Score 4) Obvious fish odor.

Score 5) Strong fish odor.

Score 6) Extremely strong fish odor.

TABLE 21

| | | Average score |
|---|---|---|
| COMP. EX. 73 | Control | 6.0 |
| COMP. EX. 74 | Solution containing 1 g of calcium | 3.4 |
| | lignosulfonate | |
| COMP. EX. 75 | Solution containing 1 g of laccase | 5.8 |
| EX. 36 | Solution containing calcium | 1.4 |
| | lignosulfonate + laccase | |

In the above table, the control means a case of water free from the calcium lignosulfonate+laccase of the Example.

### EXAMPLE 37 AND COMPARATIVE EXAMPLES 76 TO 78

### Effect of Eliminating Bleaching Odor

A cloth piece (10 cm×10 cm) was immersed in a hypochlorous acid-based bleaching agent for 2 minutes. Then the cloth was taken out and rinsed with water. Next, it was immersed in water containing the deodorant composition-containing powder detergent (0.5% by weight) prepared in Example 13. After washing for 5 minutes at room temperature, the cloth was rinsed again. Then it was sensorily evaluated by 5 skilled panelists in accordance with the following evaluation criteria.

Table 22 summarizes the obtained results.

Criteria for Evaluation:

Score 1) No bleaching odor.

Score 2) Slight bleaching odor.

Score 3) Somewhat bleaching odor.

Score 4) Obvious bleaching odor.

Score 5) Strong bleaching odor.

Score 6) Extremely strong bleaching odor.

TABLE 22

| | | Average score |
|---|---|---|
| COMP. EX. 76 | Control | 6.0 |
| COMP. EX. 77 | Powder detergent containing sodium | 3.8 |
| | lignosulfonate | |
| COMP. EX. 78 | Powder detergent containing laccase | 6.0 |
| EX. 37 | Powder detergent containing sodium | 1.2 |
| | lignosulfonate + laccase | |

In the above table, the control is a case of the powder detergent free from sodium lignosulfonate+laccase in Example.

### EXAMPLE 38 AND COMPARATIVE EXAMPLES 79 TO 81

### Effect of Eliminating Perming Odor

The following method was employed to evaluate the deodorizing effect on perming odor with the use of a shampoo containing the deodorant composition according to the invention.

1.8 g of a tress of hair to be tested was immersed in 50 mL of a perming liquid 1 (a 6% aqueous solution of thioglycolic acid adjusted to pH 9.3 with aqueous ammonia) for 30 minutes. After wiping off the perming liquid 1 adhering to the tress, the tress was washed with 100 mL of water. Then, it was immersed in 50 mL of a perming liquid 2 (a 5% aqueous solution of potassium bromate) for 20 minutes. After wiping off the perming liquid 2 adhering to the tress, the tress was immersed in 1000 mL of water containing the shampoo (1% by weight) prepared in Example 14 for 5 minutes. After wiping off the shampoo adhering to the tress, the tress was washed with 100 mL of water and then the water adhering to the tress was wiped off. Then the tress was sensorily evaluated by 4 panelists in accordance with the following evaluation criteria.

Table 23 summarizes the obtained results.

Criteria for Evaluation:

Score 1) No perming odor.

Score 2) Slight perming odor.

Score 3) Somewhat perming odor.

Score 4) Obvious perming odor.

Score 5) Strong perming odor.

Score 6) Extremely strong perming odor.

TABLE 23

| | | Average score |
|---|---|---|
| COMP. EX. 79 | Control | 5.3 |
| COMP. EX. 80 | Shampoo containing calcium | 3.1 |
| | lignosulfonate | |
| COMP. EX. 81 | Shampoo containing laccase | 5.1 |
| EX. 38 | Shampoo containing calcium | 1.0 |
| | lignosulfonate + laccase | |

In the above table, the control is a case of the shampoo free from calcium lignosulfonate+laccase in Example.

### INDUSTRIAL APPLICABILITY

The invention provides deodorant compositions having an excellent deodorizing effect on various offensive odor components. Moreover, these deodorant compositions have another advantage of giving off little foul odor derived from the substrate. Because they contain lignin as a constituent, the deodorant compositions are nondetrimental to humans and environment. From this viewpoint, they can be considered as excellent deodorants. Moreover, deodorant compositions containing water-soluble lignin chosen from among the various types of lignins can be considered as excellent deodorant materials from the viewpoint of the effective utilization of raw materials, in addition to the advantage of being nondetrimental to humans and the environment.

While the invention has been described in detail and with reference to specific embodiments thereof, it will be apparent to one skilled in the art that various changes and modifications can be made therein without departing from the spirit and scope thereof.

This application is based on Japanese patent application. No. 2002-319610 filed on Nov. 1, 2002, the entire contents thereof being hereby incorporated by reference.

1. A deodorant composition characterized by containing lignin and a phenolic compound-oxidizing enzyme.

2. A deodorant composition according to claim 1, wherein the lignin is a soluble lignin.

3. A deodorant composition according to claim 2, wherein the soluble lignin is water-soluble lignin.

4. A deodorant composition according to any one of claims 1 to 3, further containing a fragrance and/or a flavor.

5. An oral care product characterized by containing a deodorant composition according to any one of claims 1 to 3.

6. A sanitary product characterized by containing a deodorant composition according to any one of claims 1 to 3.

7. A pet product characterized by containing a deodorant composition according to any one of claims 1 to 3.

8. A food characterized by containing a deodorant composition according to any one of claims 1 to 3.

9. A feed characterized by containing a deodorant composition according to any one of claims 1 to 3.

10. Use of a deodorant composition as claimed in claim 1, in an oral care product, a sanitary product, a pet product, a food or a feed.
